(19) United States
(12) Patent Application Publication
(10) Pub. No.: US 2007/0050031 A1
(43) Pub. Date: Mar. 1, 2007

(54) Spinal implant and implant inserter

(76) Inventor: Hamid Khosrowshahi, Tarrytown, NY (US)

Correspondence Address: Epstein & Gerken;Suite 340, 1901 Research Boulevard, Rockville, MD, 20850, US

(21) Appl. No.: 11/508,561

(22) Filed: Aug. 23, 2006

Related U.S. Application Data
(60) Provisional application No. 60/710,696, filed on Aug. 23, 2005.

(51) Int. Cl. A61F 2/44; A61F 2/46

(52) U.S. Cl. 623/17.11

(57) ABSTRACT

An intervertebral implant includes an implant body having superior and inferior sides, medial and lateral sides, and anterior and posterior ends. The superior and inferior sides each include a central section and convex side sections extending from opposite sides of the central section to the medial and lateral sides. The convex side sections are composed of a plurality of convex surface segments of different radii of curvature. The medial and lateral sides each have a concave inward portion and superior and inferior outward portions defining a cavity. An implant inserter includes a pair of holding members for being removably received in the cavities of the implant with an intermediate convex section of each holding member extending between the superior and inferior sides of the implant. The intermediate convex section of each holding member is composed of a plurality of convex surface segments of different radii of curvature.

### CROSS-REFERENCE TO RELATED PATENT APPLICATION

This application claims priority from prior provisional U.S. patent application Ser. No. 60/710,696 filed Aug. 23, 2005, the entire disclosure of which is incorporated herein by reference.

### BACKGROUND OF THE INVENTION

1. Field of the Invention

The present invention relates to spinal implants and, in particular, to intervertebral implants and to inserter instruments for spinal or intervertebral implants.

2. Brief Discussion of the Related Art

Intervertebral implants have been proposed for use in spinal procedures involving the fusion of adjacent vertebrae. Intervertebral implants are typically placed between adjacent vertebrae with superior and inferior sides of the implant in engagement with the adjacent vertebrae. In some implants, the superior and inferior sides are provided with raised formations to better grip the adjacent vertebrae for enhanced stabilization of the vertebrae and resistance of the implant to displacement, subsidence and/or migration. Intervertebral implants usually have an open interior allowing for the placement of bone growth material into or through the implant as well as blood flow through the implant. It is oftentimes desirable to place more than one implant between the adjacent vertebrae in close side by side relation.

Representative intervertebral spinal implants are disclosed in U.S. Pat. No. 5,443,514 and No. 5,716,415 to Steffee, U.S. Pat. No. 5,658,337 to Kohrs et al, U.S. Pat. No. 5,904,719 to Errico et al, U.S. Pat. No. 6,440,170 B1 to Jackson, U.S. Pat. No. 6,500,206 B1 and U.S. Pat. No. 6,843,804 B2 to Bryan, U.S. Pat. No. 6,783,545 B2 to Castro et al, and U.S. Pat. No. 6,855,166 B2 to Kohrs, and in U.S. Patent Application Publications No. 2003/0018389 A1 to Castro et al, No. 2004/0230305 A1 to Gorensek et al, and Nos. 2005/0049587 A1 and 2005/0065606 A1 to Jackson.

The Steffee patents are illustrative of intervertebral spinal implants that have triangular teeth for engaging the adjacent vertebrae rotated to an implanted orientation between the adjacent vertebrae via an inserter instrument. The implants of the Steffee patents have rectangular cross-sectional configurations with flat parallel medial and lateral sides to extend between the adjacent vertebrae when the implants are in the implanted orientation, and straight inferior and superior sides having the teeth thereon. The rectangular cross-sectional configuration of the implants presents resistance to rotation between the adjacent vertebrae and thusly makes it more difficult to rotate the implants to the implanted orientation via the inserter instrument. Furthermore, as the implants are rotated between the adjacent vertebrae, their longer diagonal cross-sectional dimension must pass between the adjacent vertebrae such that the vertebrae are undesirably distracted considerably more than is necessary for the sides of the implants to fit between the adjacent vertebrae.

The implant shown in the Kohrs et al patent has ridges along its superior and inferior sides for engaging the adjacent vertebrae in an implanted orientation but presents many of the same disadvantages as the Steffee implants with respect to being rotated between the adjacent vertebrae. The ridges have a concave configuration and engage with the prongs of an insertion tool. The implant engaged with the insertion tool presents a cylindrical surface for insertion of the implant into a round bore formed between the adjacent vertebrae. The implant is inserted in the round bore longitudinally or rotatably by threading the implant into the bore.

The Errico et al patent relates to an implant having parallel upper and lower surfaces that are continuously flat between the side walls of the implant. Ridges extend outwardly from the flat upper and lower surfaces for engaging the adjacent vertebrae when the implant is rotated between the adjacent vertebrae to an implanted orientation. The dimensions and cross-sectional configuration of the implant causes overdistraction of the adjacent vertebrae when the implant is rotated to the implanted orientation.

The Kohrs and Jackson patents and the Jackson patent application publications pertain to implants having an external thread and to insertion tools having threads complementary to those of the implants for threaded insertion between adjacent vertebrae. Spinal implants having an external thread are also disclosed in the Castro et al patent and in the Castro et al patent application publication.

The Bryan patents are illustrative of spinal implants having dimensions and cross-sectional configurations intended to deliberately maximize distraction of adjacent vertebrae when the implants are driven longitudinally between the adjacent vertebrae. The Gorensek et al patent application publication is representative of intervertebral implants having continuously curving upper and lower surfaces and having flat parallel side surfaces.

### SUMMARY OF THE INVENTION

The present invention is generally characterized in an intervertebral implant comprising an implant body having superior and inferior sides, opposed medial and lateral sides, an anterior end wall, and a posterior end wall. The implant body may include an anterior interior compartment and a posterior interior compartment separated by an interior partition. The interior partition may extend between and connect the superior, inferior and medial and lateral sides. A passage may extend through the partition to establish communication between the compartments within the interior of the implant body. Anterior and posterior windows may be provided in each of the superior and inferior sides to establish communication with the respective anterior and posterior compartments through the superior and inferior sides. Anterior and posterior openings may be provided in each of the medial and lateral sides to establish communication with the respective anterior and posterior compartments through the medial and lateral sides. Bore holes may be provided through the anterior and posterior end walls to communicate respectively with the anterior and posterior compartments. The superior and inferior sides may carry parallel ridges that extend perpendicular to a central longitudinal axis of the implant body. The outer surfaces of the superior and inferior sides each have a straight or level central section and convexly curving side sections extending from opposite sides of the central section to the medial and lateral sides. Each side section is composed of a plurality of convex surface segments of different radii of curvature. The outer surfaces of the medial and lateral sides each have a concave inward portion between superior and inferior concave outward portions that curve in opposition to the inward portion and meet the outer surfaces of the respective superior and inferior sides. The outer surfaces of the medial and lateral sides circumscribe cavities disposed on opposite sides of the implant body and extending lengthwise therealong to matingly receive respective holding members of an implant inserter according to the present invention.

The implant inserter is generally characterized in an elongate shaft structure having a distal end and a proximal end, a handle at the proximal end of the shaft structure, and an implant holding device at the distal end of the shaft structure for removably holding the implant. The implant holding device comprises a pair of longitudinally elongate holding members for being respectively received with a mating fit in the cavities of the implant. Each holding member has a cross-sectional configuration defined by an external surface including a convex inner portion having a configuration matching or complementary to the concave inward portion of the corresponding medial or lateral side of the implant and a convex outer portion curving in opposition to the convex inner portion and having superior and inferior convex sections matching or complementary to the superior and inferior concave outward portions of the corresponding medial or lateral side of the implant. Each convex outer portion of the holding members also includes an intermediate convex section extending between the superior and inferior convex sections. The intermediate convex sections extend between the side sections of the superior and inferior sides of the implant when the holding members are received in the corresponding cavities of the implant. The intermediate convex sections of the holding members are each composed of a plurality of convex surface segments of different radii of curvature and cooperate with the convex side sections of the implant to present a smooth, variably curving convex configuration when the implant is assembled to the inserter with the holding members respectively matingly received in the cavities of the implant for use during a spinal procedure.

### BRIEF DESCRIPTION OF THE DRAWINGS

FIG. 1 is a perspective view of an intervertebral implant according to the present invention.

FIG. 2 is a top view of the implant.

FIG. 3 is a side view of the implant.

FIG. 4 is a posterior end view of the implant.

FIG. 5 is an anterior end view of the implant.

FIG. 6 is a sectional view taken along line 6-6 of FIG. 3.

FIG. 7 is a sectional view taken along line 7-7 of FIG. 5.

FIG. 8 is a sectional view taken along line 8-8 of FIG. 3.

FIG. 9 is an enlarged view of detail A of FIG. 7.

FIG. 10 is an enlarged view of detail B of FIG. 7.

FIG. 11 is a side view of an implant inserter according to the present invention showing the implant assembled to the inserter.

FIG. 12 is a top view of the implant inserter with the implant assembled thereto.

FIG. 13 is an enlarged view of detail C of FIG. 11.

FIG. 14 is an enlarged side view depicting the holding device of the inserter with the implant held by holding members of the holding device.

FIG. 15 is a sectional view taken along line 15-15 of FIG. 14.

FIG. 16 is an enlarged view of FIG. 15.

FIG. 17 is a broken sectional view of the implant inserted by the inserter in an initial orientation between adjacent vertebrae.

FIG. 18 is a broken sectional view of the implant rotated by the inserter to an implanted orientation between the adjacent vertebrae.

### DESCRIPTION OF THE PREFERRED EMBODIMENTS

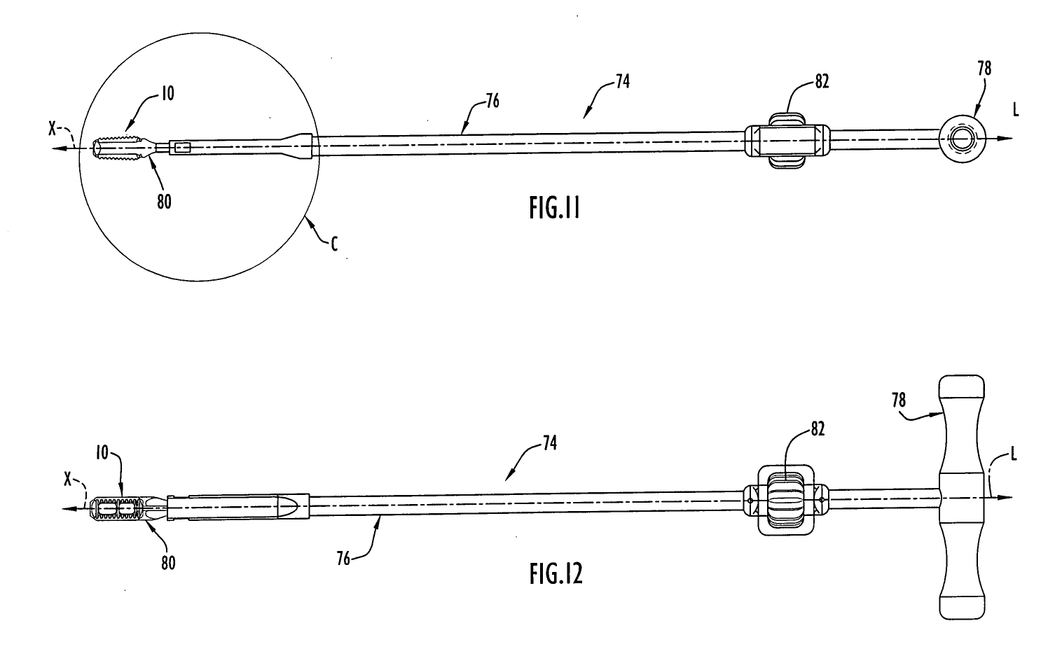

An intervertebral implant 10 according to the present invention is depicted in FIGS. 1-10. The implant 10 comprises an implant body 12 including superior (top) and inferior (bottom) sides 14, opposed medial and lateral sides 16 connected to the superior and inferior sides, an anterior end wall 18 connected to the superior, inferior, and medial and lateral sides, a posterior end wall 19 connected to the superior, inferior, and medial and lateral sides, and a central longitudinal axis X. The superior and inferior sides 14 are inverted images of each other. The medial and lateral sides 16 are mirror images of each other.

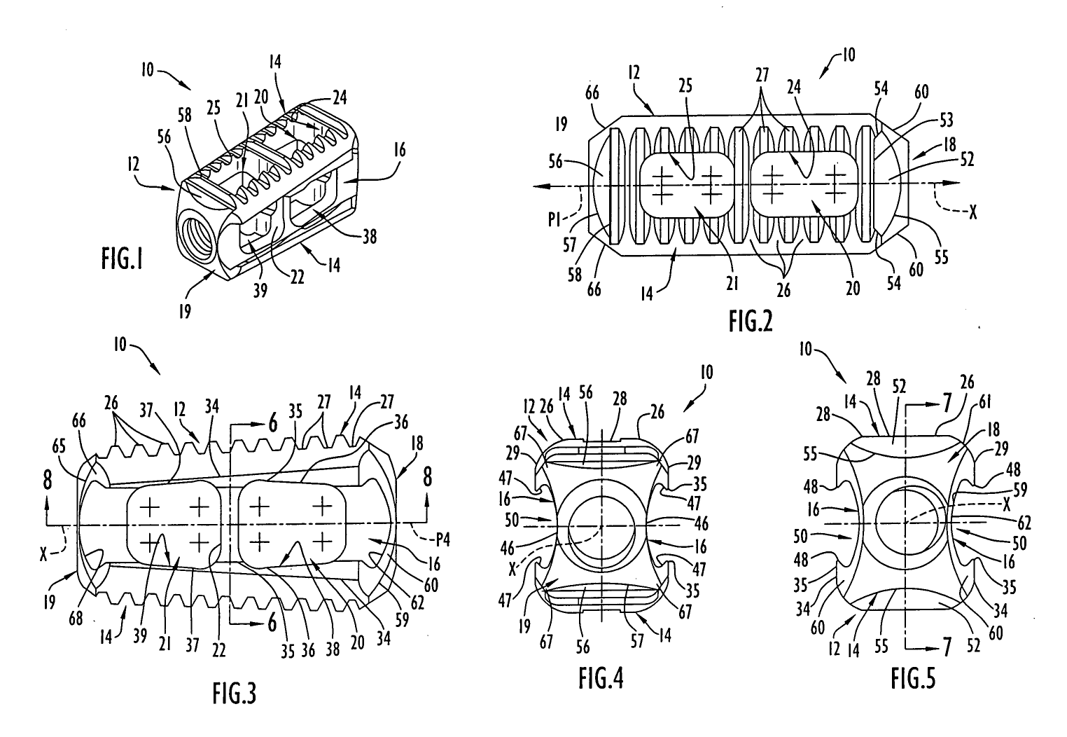

The body 12 includes an anterior interior compartment 20 and a posterior interior compartment 21 separated by an internal partition 22 connecting the superior, inferior, and medial and lateral sides. A passage 23 extends through the partition 22 coaxial with the central longitudinal axis X and establishes communication between compartments 20 and 21 within the interior of the body 12. An anterior window 24 and a posterior window 25 are formed through each of the superior and inferior sides 14 respectively in communication with the anterior and posterior interior compartments 20 and 21. The anterior windows 24 are located between the anterior end wall 18 and the partition 22. The posterior windows 25 are located between the posterior end wall 19 and the partition 22. The anterior and posterior windows 24 and 25 and the anterior and posterior interior compartments 20 and 21 are bisected longitudinally by a plane P1 containing the central longitudinal axis X as shown in FIG. 2. The anterior windows 24 and anterior interior compartment 20 are greater in length than the posterior windows 25 and posterior interior compartment 21. The anterior and posterior windows 24 and 25 have a generally rectangular peripheral configuration with radiused or arcuate corners.

Outer surfaces of each of the superior and inferior sides 14 are formed with a plurality of parallel ridges 26 alternating with parallel grooves 27 along the length of the implant body 12. The ridges 26 and grooves 27 extend in a direction perpendicular to the central longitudinal axis X, with some of the ridges and grooves being discontinuous due to the windows 24 and 25. The ridges 26, which have crest surfaces formed by the outer surfaces of the superior and inferior sides 14, and the grooves 27 are explained further below.

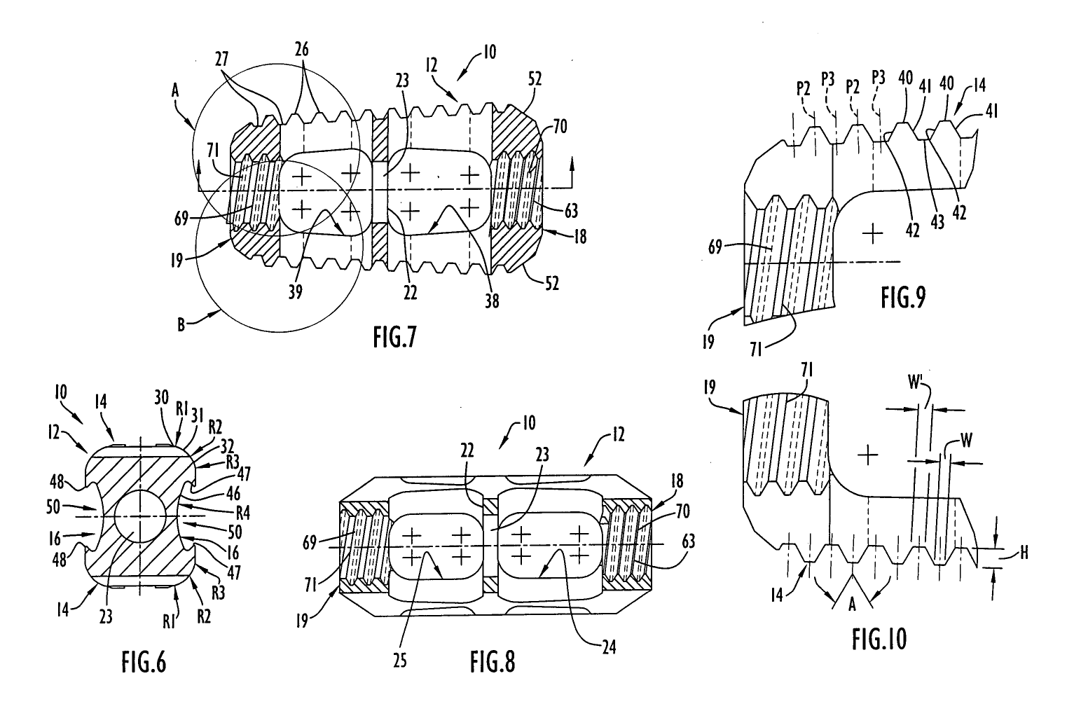

The outer surfaces of the superior and inferior sides 14 each have a straight or level central section 28 and convex side sections 29 extending from opposite sides of the central section 28 to the respective medial and lateral sides 16. Each convex side section 29 is composed of a plurality of convex surface segments of different radii of curvature. As shown in FIG. 6, each convex side section 29 comprises a first convex surface segment 30 extending from the central section 28 toward the corresponding convex side section 29 of the opposite side 14 with a radius of curvature R1, a second convex surface segment 31 extending from the first convex surface segment 30 toward the corresponding convex side section 29 of the opposite side 14 with a radius of curvature R2, and a third convex surface segment 32 extending from the second convex surface segment 31 toward the corresponding convex side section 29 of the opposite side 14 with a radius of curvature R3 to meet the outer surface of the corresponding medial or lateral side 16. Accordingly, both convex side sections 29 of the superior side 14 curve toward the corresponding convex side sections 29 of the inferior side 14 and vice versa.

The second convex surface segments 31 meet the third convex surface segments 32 at straight edges 34 extending from anterior to posterior. The third convex surface segments 32 meet the outer surfaces of the corresponding sides 16 at edges or tips 35 extending from anterior to posterior. Each edge 35 has anterior and posterior recesses or cut-outs 36 and 37 in respective correspondence with anterior and posterior openings 38 and 39 in the corresponding medial or lateral side 16 as described further below.

The superior and inferior sides 14 are angled inwardly toward one another from anterior to posterior such that the height of the implant 10 between the outer surfaces of the superior and inferior sides 14 in a direction perpendicular to central longitudinal axis X tapers or decreases from anterior to posterior. The width of the implant between the third convex surface segments 32 in a direction perpendicular to the height and to the central longitudinal axis X is uniform or constant along the length of the third convex surface segments 32 from anterior to posterior. In addition, the third convex surface segments 32 have a depth between the edges 34 and 35 which tapers or decreases from anterior to posterior. The height of the implant 10 is selected so that the ridges 26 of the superior and inferior sides 14 will cut into and be in engagement with facing surfaces of adjacent vertebrae when the implant 10 is placed in an implanted orientation between the adjacent vertebrae as explained further below.

The configuration of the ridges 26 and the grooves 27 is best shown in FIGS. 9 and 10. Each ridge 26 is bisected by a ridge plane P2, the ridge planes P2 being parallel and equally spaced along the length of the superior and inferior sides 14. Each groove 27 is bisected by a groove plane P3, and the groove planes P3 are also parallel and equally spaced along the length of the superior and inferior sides 14 as well as being parallel to the ridge planes P2. Each ridge 26 has a crest surface 40 formed by the outer surface of the corresponding superior or inferior side 14, a front surface 41 extending angularly downwardly and outwardly from a front edge of the crest surface 40, and a back surface 42 extending angularly downwardly and outwardly from a back edge of the crest surface 40 at the same angle as but in opposition to the front surface 41. Except for the anteriormost groove and the posteriormost groove, each groove 27 has a base surface 43 recessed from the crest surface 40, a forward surface formed by the back surface 42 of the adjacent anterior ridge 26, and a rearward surface formed by the front surface 41 of the adjacent posterior ridge 26. The ridges 26 have a height H between the crest surfaces 40 and the base surfaces 43 as shown in FIG. 10, and the height H is also the depth of grooves 27. Except for the anteriormost and the posteriormost grooves, the ridges 26 and the grooves 27 are of the same uniform height. In the case of the anteriormost groove 27, as shown in FIGS. 3 and 7, the forward surface of the groove extends upwardly from its base surface a lesser distance than its rearward surface. In the case of the posteriormost groove 27, the rearward surface thereof extends upwardly from its base surface a lesser distance than its forward surface. As illustrated in FIG. 10, the intersecting planes containing the front and back surfaces 41 and 42 of each ridge 26 define an included angle A, which may be an angle of or about 60°. The width W of each crest surface 40 may be slightly less than the width W′ of each base surface 43. For example, in a preferred but not limiting embodiment, the width W may be 0.015 inch and the width W′ may be 0.020 inch. The height H of the ridges and the depth of the grooves may be 0.026 inch in the preferred but not limiting embodiment.

The ridge planes P2 are norrtial to the crest surfaces 40, and the groove planes P3 are normal to the base surfaces 43. Since the outer surfaces of the superior and inferior sides 14 are angled inwardly toward one another from anterior to posterior, the ridge and groove planes are angled, sloped or canted in a posterior direction and are not perpendicular to central longitudinal axis X as best understood from FIGS. 9 and 10. Accordingly, the ridge and groove planes for the superior side 14 are angled, sloped or canted in opposition to the ridge and groove planes for the inferior side 14 due to the tapered height of the implant 10 and the inward angle of the superior and inferior sides 14 from anterior to posterior. The ridge and groove planes define an acute angle with the central longitudinal axis X. The ridges 26 and grooves 27 extend along the central sections 28 and partly along the convex side sections 29 of the superior and inferior sides 14, the grooves 27 terminating at tapered outer ends along the side sections 29.

The outer surfaces of the medial and lateral sides 16 each have a concave inward portion 46 between superior and inferior concave outward portions 47 which meet the outer surfaces of sides 14 at the edges or tips 35. The concave inward portions 46 have a radius of curvature R4 and curve in the direction of the central longitudinal axis X. The concave outward portions 47 are reversely curved from their inward portions 46 and curve in opposition to their inward portions in a direction away from the central longitudinal axis X. The superior outward portions 47 extend toward the corresponding inferior outward portions 47 and vice versa. The superior and inferior outward portions 47 on each side 16 meet the corresponding third convex surface segments 32 at the edges or tips 35 to form superior and inferior overhanging lips or protrusions 48 on each side 16 of the body 12. The outer surfaces of sides 16 between the corresponding edges 35 respectively circumscribe cavities 50 on the opposite sides of the body 12 and extending lengthwise along the implant body 12.

Each side 16 has the anterior opening 38 formed therethrough between the anterior end wall 18 and the partition 22 and has the posterior opening 39 formed therethrough between the posterior end wall 19 and the partition 22. The anterior openings 38 establish communication through the sides 16 with the anterior interior compartment 20, and the posterior openings 39 establish communication through the sides 16 with the posterior interior compartment 21. The anterior and posterior openings 38 and 39 are bisected longitudinally by a plane P4 containing the central longitudinal axis X as depicted in FIG. 3. The anterior and posterior openings 38 and 39 have a generally oblong peripheral configuration with radiused or arcuate corners, the anterior openings 38 being greater in length than the posterior openings 39. The anterior openings 38 taper or decrease in width in a direction away from the partition 22 toward the anterior end of the implant body 12. The posterior openings 39 taper or decrease in width in a direction away from the partition 22 toward the posterior end of the implant body 12. The anterior and posterior cutouts 36 and 37 in the edges 35 respectively conform to and follow upper and lower peripheral edge portions of the anterior and posterior openings 38 and 39 as best seen in FIG. 3. At the anterior and posterior ends of the implant body 12, the medial and lateral sides 16 terminate at arcuate edges which are the outward arcuate edges of medial and lateral connecting surfaces as explained further below.

At the anterior end of the implant body 12, the outer surfaces of the superior and inferior sides 14 are connected to respective superior and inferior anterior transition surfaces 52 that slope angularly inwardly toward one another from the respective outer surfaces of the superior and inferior sides 14 to the anterior end wall 18. The superior and inferior anterior transition surfaces 52 meet the outer surfaces of the respective superior and inferior sides 14 at straight edges 53 perpendicular to the central longitudinal axis X and at diagonal edges 54 extending outwardly in opposite medial and lateral directions from the opposite medial and lateral sides of the straight edge 53. The superior and inferior anterior transition surfaces 52 meet the anterior end wall 18 at respective arcuate edges 55 connecting the diagonal edges 54. At the posterior end of the implant body 12, the outer surfaces of the superior and inferior sides 14 are respectively joined to the posterior end wall 19 by superior and inferior posterior transition surfaces 56 that slope angularly inwardly toward one another from the respective outer surfaces of the superior and inferior sides 14 to the posterior end wall 19. The superior and inferior posterior transition surfaces 56 are similar to the superior and inferior anterior transition surfaces 52 except that the arcuate edges 57 of the posterior transition surfaces 52 are connected to the straight edges 58 without any diagonal edges and, also, the radius of curvature for the arcuate edges 57 of the posterior transition surfaces 56 is smaller than the radius of curvature for the arcuate edges 55 of the anterior transition surfaces 52. The outer surfaces of the superior and inferior sides 14 are also connected to corner extensions of the anterior and posterior end walls 18 and 19 and to medial and lateral anterior and posterior connecting surfaces as described further below.

The anterior end wall 18 is bordered superiorly and inferiorly by the arcuate edges 55 of the superior and inferior anterior transition surfaces 52 and by the diagonal edges 54 of the superior and inferior anterior transition surfaces 52. The anterior end wall 18 is bordered medially and laterally by inward arcuate edges 59 of the medial and lateral anterior connecting surfaces 60. As depicted in FIG. 5, the arcuate edges 55 of the superior and inferior anterior transition surfaces 52 are concavely curved toward the central longitudinal axis X, and the inward arcuate edges 59 of the medial and lateral anterior connecting surfaces 60 are concavely curved toward the central longitudinal axis X such that the anterior end wall 18 defines four corner extensions 61. The two superior corner extensions 61 are joined to the outer surface of the superior side 14 at straight edges, and the two inferior corner extensions 61 are similarly joined to the outer surface of the inferior side 14 at straight edges. The medial and lateral anterior connecting surfaces 60 have concavely curved outward arcuate edges 62 where the connecting surfaces 60 meet the outer surfaces of the medial and lateral sides 16. A bore 63 extends through the anterior end wall 18 coaxial with the central longitudinal axis X and establishes communication with the anterior interior compartment 20. The anterior end wall 18 is planar around or circumscribing bore 63 and transitions from being planar to having a posterior slope or angle extending superiorly and inferiorly toward the superior and inferior sides 14 and extending medially and laterally toward the medial and lateral sides 16. Also, the medial and lateral anterior connecting surfaces 60 are angled in a posterior direction from the plane of the anterior end wall 18 as best seen with reference to FIGS. 2 and 3.

As seen in FIG. 4, the posterior end wall 19 is similar to the anterior end wall 18 and is bordered superiorly and inferiorly by the arcuate edges 57 of the superior and inferior posterior transition surfaces 56 and medially and laterally by the inward arcuate edges 65 of the medial and lateral posterior connecting surfaces 66 to define four corner extensions 67 joined to the outer surfaces of the superior and inferior sides 14 at straight edges as described for anterior end wall 18. The outer surfaces of the superior and inferior sides 14 meet the medial and lateral posterior connecting surfaces 66 at outward arcuate edges 68 of the connecting surfaces 66. A bore 69 extends through the posterior end wall 19 coaxial with the central longitudinal axis X and establishes communication with the posterior interior compartment 21. The posterior end wall 19 is planar around or circumscribing bore 69 but transitions from being planar to having an anterior slope or angle extending superiorly and inferiorly toward the superior and inferior sides 14 and medially and laterally toward the medial and lateral sides 16. In addition, the medial and lateral posterior connecting surfaces 66 are angled in the anterior direction from the plane of the posterior end wall 19. Accordingly, as shown in FIG. 2, the angled medial and lateral anterior and posterior connecting surfaces 60 and 66 impart a beveled configuration to the corners of the implant body 12. The bores 63 and 69 are respectively provided with internal thread formations 70 and 71 as seen in FIGS. 7-10.

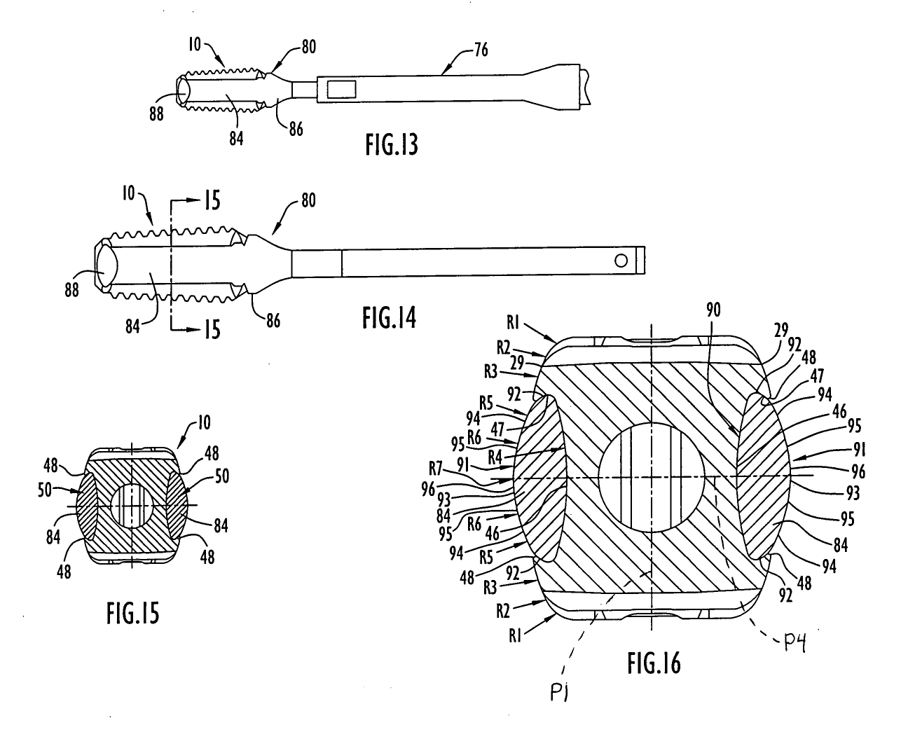

An implant inserter 74 for use in a spinal procedure to implant the implant 10 between adjacent vertebrae is shown in FIGS. 11-16 with the implant 10 assembled to the inserter 74. The inserter 74 includes an elongate shaft structure 76 having a distal end, a proximal end and a central longitudinal axis L, a handle 78 at the proximal end of the shaft structure 76, and a holding device 80 at the distal end of the shaft structure 76 for releasably or removably holding the implant 10. The inserter 74 can also include an operating member 82, such as a rotatable knob, for rotating the holding device 80 to rotate the implant 10 as explained further below. The shaft structure 76 may include a single shaft or a plurality of concentrically arranged shafts for mounting holding device 80 and, optionally, for effecting rotation of the holding device 80 in response to operation of the operating member 82. The holding device 80 comprises a pair of elongate holding members 84 extending longitudinally in parallel in the distal direction from a hub 86 to respective distal tips 88 having an inward taper. The holding members 80 are disposed in spaced relation on opposite sides of the central longitudinal axis L to be slidably inserted with a mating fit into the cavities 50 of the implant 10.

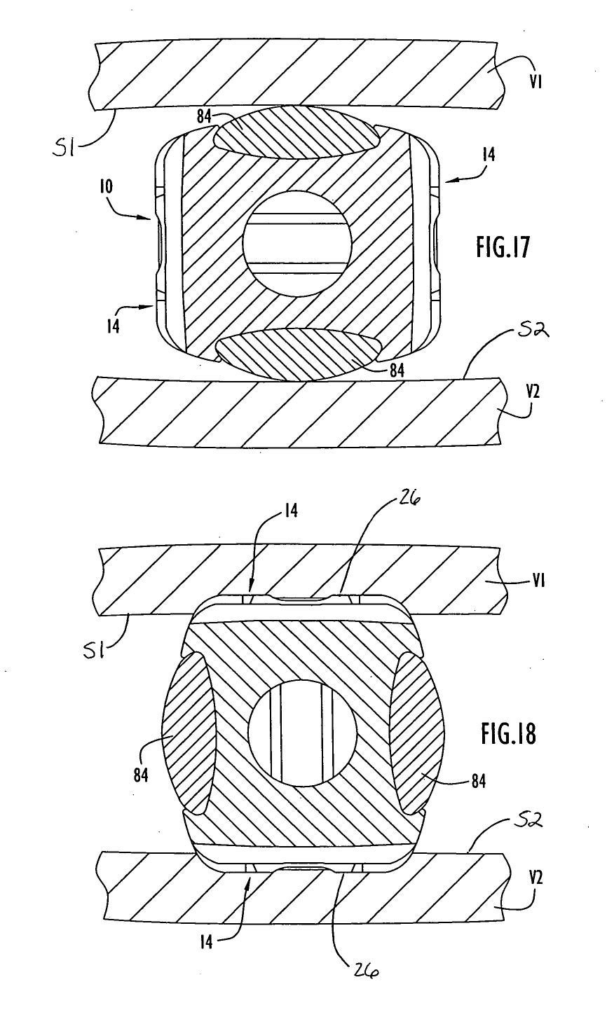

As best shown in FIGS. 15 and 16, each holding member 80 has a cross-sectional configuration extending lengthwise along the holding member defined by an external surface including a convex inner portion 90 that matches or is complementary to the concave inward portion 46 of the implant 10 and a convex outer portion 91 curving in opposition to the inner portion 90. The convex inner portion 90 has a radius of curvature R4 complementary to the concave inward portion 46 of the implant 10. The convex outer portion 91 comprises superior and inferior convex sections 92 matching or complementary to the configuration of the superior and inferior concave outward portions 47 of the implant 10 to mate with the cavity 50, and an intermediate convex section 93 extending between the superior and inferior convex sections 92. The holding members 84 are able to be removably inserted into the cavities 50 of implant 10 in mating engagement by sliding the holding members 84, distal tips first, into the cavities 50 until the distal tips 88 of the holding members 84 are adjacent the end of the implant 10. When the holding members 84 are matingly received in the cavities 50, the lips or protrusions 48 overlap the convex outer portions 91 of the holding members 84 such that the implant 10 is restricted from moving relative to the holding members 84 in a direction radial to the central longitudinal axes X and L. The intermediate convex section 93 of each holding member 84 extends between the corresponding lips 48 and has a convex curvature composed of a plurality of convex surface segments of different radii of curvature.

As seen in FIG. 16, each intermediate convex section 93 is composed of upper and lower convex surface segments 94 extending toward each other with a radius of curvature R5, intervening convex surface segments 95 extending from the upper and lower convex surface segments 94 with a radius of curvature R6, and a middle convex surface segment 96 extending between the intervening convex surface segments 95 with a radius of curvature R7. The convex surface segments 94, 95 and 96 cooperate with the first, second and third convex surface segments 30, 31 and 32 of the side sections 29 of the implant to present a smooth, variably curving convex configuration along the opposite sides of the implant 10 when the implant 10 is held by the holding members 84 matingly received in the cavities 50.

The radius of curvature R1 of the first convex surface segments 30 is greater than the radius of curvature R2 of the second convex surface segments 31. The radius of curvature R3 of the third convex surface segments 32 is greater than the radius of curvature R1. The radius of curvature R5 of the upper and lower convex surface segments 94 is less than the radius of curvature R2. The radius of curvature R6 of the intervening convex surface segments 95 is less than the radius of curvature R5. The radius of curvature R7 of the middle convex surface segments is less than the radius of curvature R6. In a preferred but not limiting embodiment, the radius of curvature R1 for the first convex surface segments 30 is in the range of 0.2 to 0.6 inch; the radius of curvature R2 for the second convex surface segments 31 is in the range of 0.1 to 0.35 inch; the radius curvature R3 for the third convex surface segments 32 is in the range of 1.00 to 3.5 inches; the radius of curvature R4 for the concave inward portions 46 of the medial and lateral sides 16 and the convex inner portions 90 of the holding members 84 is in the range of 0.2 to 0.4 inch; the radius of curvature R5 for the upper and lower convex surface segments 94 is in the range of 0.1 to 0.35 inch; the radius of curvature R6 for the intervening convex surface segments 95 is in the range of 0.1 to 0.35 inch; and the radius of curvature R7 for the middle convex surface segments 96 is in the range of 0.1 to 0.35 inch. In one preferred embodiment, for example, R1 is 0.400 inch, R2 is 0.220 inch, R3 is 2.250 inches, R4 is 0.305 inch, R5 is 0.217 inch, R6 is 0.211 inch, and R7 is 0.206 inch.

During a spinal procedure, the implant inserter 74 is used to place the implant 10 between adjacent vertebrae with the implant 10 in an initial orientation angularly displaced or rotated 90° or substantially 90° from an implanted orientation. When the implant is placed by the inserter 74 between the adjacent vertebrae V1 and V2 in the initial orientation as illustrated in FIG. 17, the convex outer portions 91 of the holding members will respectively face the adjacent vertebrae V1 and V2. The inward taper of the distal tips 88 of the holding member 84 provide the inserter 74 with a lead-in angle which allows for gentle distraction of the vertebrae V1 and V2 and surrounding soft tissue to facilitate introduction of the inserter 74, distal tips first, and the implant 10 held by the inserter 74, into the intervertebral space between the vertebrae V1 and V2. After placement of the implant 10 in the initial orientation at a selected location between the adjacent vertebrae V1 and V2, the holding device 80 is rotated 90° or substantially 90° to correspondingly rotate the implant 10 so that the implant 10 is in its implanted orientation with the superior and inferior sides 14 of the implant 10 respectively facing and in engagement with bone of the adjacent vertebrae V1 and V2 as shown in FIG. 18. By providing the operating member 82 to effect rotation of the holding device 80, the holding members 84 and implant 10 can be rotated without rotating the entire inserter. If it is desired to relocate the implant 10 to a more optimal position between the vertebrae V1 and V2, the holding device 80 can be rotated 90° or substantially 90° to correspondingly rotate the implant 10 from the implanted orientation back to the initial orientation, allowing the implant 10 to be moved to another location between the vertebrae V1 and V2 prior to again rotating the implant 10 to the implanted orientation. Once the implant 10 is in the implanted orientation at an optimal location, the holding members 84 are slidably withdrawn from the cavities 50 of the implant 10 to detach the inserter 74 from the implant 10 leaving the implant 10 in place between the vertebrae V1 and V2. It should be appreciated that the inserter 74 can be detached from the implant 10 after rotating the implant 10 to the implanted orientation and then be reattached to the implant 10 for “derotating” the implant 10 from the implanted orientation back to the initial orientation for repositioning or removal of the implant 10.

The lead-in angle of the inserter 74 and the smooth, variably curving convex configuration formed by the intermediate convex sections 93 of the holding members 84 and the convex side sections 29 of the superior and inferior sides 14 of the implant 10 avoid trauma to anatomical tissue when the implant is inserted, located and rotated between the adjacent vertebrae V1 and V2 and facilitate repositioning or relocating of the implant 10, if required, by “derotating” the implant 10 via rotation of the holding members 84 and sliding the implant 10 to a different location between the vertebrae V1 and V2 prior to again rotating the implant 10 to reengage the superior and inferior sides 14 of the implant with the adjacent vertebrae V1 and V2. When the implant 10 is initially inserted and placed between the vertebrae V1 and V2 in the initial orientation, the convex surfaces of the holding members 84 facing the vertebrae V1 and V2 ensure that the vertebrae V1 and V2 are gently, non-traumatically distracted only to the extent necessary for the holding device 80 to be accommodated between the vertebrae V1 and V2. Accordingly, the vertebrae V1 and V2 need only be distracted to the extent necessary for the dimension of the holding device 80 along plane P4 to fit between the vertebrae V1 and V2. Rotational movement of the holding device 80 within the intervertebral space in either the clockwise or counterclockwise direction from the initial orientation is facilitated by the convex surfaces of the holding members 84 facing the vertebral surfaces S1 and S2 of vertebrae V1 and V2. The vertebral surfaces S1 and S2 that respectively face the convex surfaces of the holding members 84 extend in a tangential or substantially tangential direction to the convex surfaces of the holding members 84 as seen in FIG. 17. The convex surfaces of the holding members 84 adjacent or in contact with the surfaces S1 and S2 provide minimal resistance to rotation of the holding device 80 within the intervertebral space in either the clockwise or counterclockwise direction, thereby easing rotation of the holding device 80 to correspondingly rotate the implant from the initial orientation to the implanted orientation. As the holding device 80 is rotated in the intervertebral space, the convex side sections 29 of the implant 10 come into contact with the respective vertebral surfaces S1 and S2, thereby facilitating and minimizing resistance to continued rotation of the holding device 80 and implant 10 toward the implanted orientation. In addition, rotational movement of the holding device 80 and implant 10 in the intervertebral space from the initial orientation to the implanted orientation creates gentle distraction of the adjacent vertebrae V1 and V2 and surrounding soft tissue by the convex surfaces of the holding members 84 and the convex side sections 29 of the implant 10. As the ridges 26 along the superior and inferior sides 14 of the implant 10 are rotated into contact with the respective vertebral surfaces S1 and S2, the ridges 26 penetrate or cut into the vertebral surfaces S1 and S2 as the implant 10 is rotated to the implanted orientation as seen in FIG. 18. The holding device 80 and attached implant 10 combine to present an external configuration in cross-section between the vertebrae V1 and V2 which, when rotated, is similar to rotation of a circle such that the vertebrae V1 and V2 are not excessively distracted or overdistracted when the implant 10 is rotated by the holding device 80 from the initial orientation to the implanted orientation. However, rotation of the holding device 80 and attached implant 10 from the initial orientation to the implanted orientation defines a multi-arc path different from circular rotation.

Inasmuch as the present invention is subject to many variations, modifications and changes in detail, it is intended that all subject matter discussed above or shown in the accompanying drawings be interpreted as illustrative only and not be taken in a limiting sense.

What is claimed is:

1. An intervertebral implant and implant inserter, comprising
an intervertebral implant comprising an implant body including superior and inferior sides, medial and lateral sides, an anterior end and a posterior end, each of said superior and inferior sides having a central section and convexly curving sides sections on opposite sides of said central section, each of said side sections being composed of a plurality of convex surface segments of different radii of curvature, each of said medial and lateral sides having a concave inward portion defining a cavity; and
an implant inserter comprising a pair of holding members configured to be respectively removably matingly received in said cavity of said implant, each of said holding members having a cross-sectional configuration defined by a convex inner portion complementary to the corresponding one of said concave inward portions of said implant and a convex outer portion including an intermediate convex section extending between said superior and inferior sides of said implant when said holding members are respectively removably matingly received in said cavities, each of said intermediate convex sections being composed of a plurality of convex surface segments of different radii of curvature

2. The intervertebral implant and implant inserter recited in claim 1 wherein said central section of each of said superior and inferior sides is level between said convexly curving side sections.

3. The intervertebral implant and implant inserter recited in claim 2 wherein said implant body has a central longitudinal axis and each of said superior and inferior sides includes a plurality of ridges extending in a direction perpendicular to said central longitudinal axis along said central section and said convexly curving side sections of said superior and inferior sides.

4. The intervertebral implant and implant inserter recited in claim 1 wherein each of said medial and lateral sides further includes superior and inferior concave outward portions that extend from and curve in opposition to said concave inward portion, each of said superior and inferior concave outward portions meeting a corresponding one of said convexly curving side sections of said superior and inferior sides.

5. The intervertebral implant and implant inserter recited in claim 4 wherein each of said convex outer portions of said holding members includes superior and inferior convex sections extending from said intermediate convex section and being complementary to the corresponding ones of said superior and inferior concave outward portions of said implant.

6. The intervertebral implant and implant inserter recited in claim 1 wherein said implant body further includes a central longitudinal axis, an anterior interior compartment, a posterior interior compartment, an internal partition between said anterior interior compartment and said posterior interior compartment, a passage through said partition coaxial with said central longitudinal axis, an anterior bore extending through said anterior end of said implant body coaxial with said central longitudinal axis and in communication with said anterior interior compartment, and a posterior bore extending through said posterior end of said implant body coaxial with said central longitudinal axis and in communication with said posterior interior compartment.

7. The intervertebral implant and implant inserter recited in claim 6 wherein said implant body further includes an anterior window in each of said superior and inferior sides respectively in communication with said anterior interior compartment, and a posterior window in each of said superior and inferior sides respectively in communication with said posterior interior compartment.

8. The intervertebral implant and implant inserter recited in claim 7 wherein said implant body further includes an anterior opening in each of said medial and lateral sides respectively in communication with said anterior interior compartment, and a posterior opening in each of said medial and lateral sides respectively in communication with said posterior interior compartment.

9. An intervertebral implant and implant inserter, comprising
an intervertebral implant comprising an implant body including superior and inferior sides, opposed medial and lateral sides, an anterior end wall, and a posterior end wall, each of said superior and inferior sides having a straight central section and convexly curving side sections on opposite sides of said central section, each of said side sections being composed of a plurality of convex surface segments of different radii of curvature, each of said medial and lateral sides having a concave inward portion and superior and inferior concave outward portions defining a cavity, said superior and inferior concave outward portions of each of said medial and lateral sides being curved in opposition to the corresponding one of said concave inward portions and extending toward one another to meet a corresponding one of said side sections of said superior and inferior sides to respectively define superior and inferior protrusions over the corresponding one of said cavities; and
an implant inserter comprising a pair of holding members configured to be respectively removably received in said cavities with a mating fit, each of said holding members having a cross-sectional configuration defined by a convex inner portion complementary to the corresponding one of said concave inward portions of said implant, and a convex outer portion curving in opposition to said inner portion, each of said convex outer portions including superior and inferior convex sections and an intermediate convex section extending between said superior and inferior convex section, said superior and inferior convex sections being complementary to the corresponding ones of said superior and inferior concave outward portions of said implant to be respectively overlapped by said superior and inferior protrusions of said implant when said holding member is matingly received in the corresponding one of said cavities, each of said intermediate convex sections extending between the corresponding ones of said superior and inferior protrusions when said holding member is matingly received in the corresponding one of said cavities, each of said intermediate convex sections being composed of a plurality of convex surface segments of different radii of curvature.

10. The intervertebral implant and implant inserter recited in claim 9 wherein said implant body further includes a central longitudinal axis, a plurality of ridges on each of said superior and inferior sides uniformly spaced from one another between said anterior end wall and said posterior end wall, said ridges extending entirely along said central section and partly along said convexly curving side sections of each of said superior and inferior sides in a direction perpendicular to said central longitudinal axis.

11. The intervertebral implant and implant inserter recited in claim 10 wherein said ridges are respectively bisected by ridge planes disposed in parallel relation, said ridge planes being angled toward said posterior end wall at an acute angle to said central longitudinal axis.

12. The intervertebral implant and implant inserter recited in claim 11 wherein said implant body further includes a plurality of grooves on each of said superior and inferior sides in alternating arrangement with said ridges, said grooves being respectively bisected by groove planes in parallel with said ridge planes.

13. The intervertebral implant and implant inserter recited in claim 12 wherein said superior and inferior sides are angled inwardly toward one another between said anterior end wall and said posterior end wall, said ridge planes and said groove planes for said ridges and said grooves of said superior side being angled in opposition to said ridge planes and said groove planes for said ridges and said grooves of said inferior side.

14. The intervertebral implant and implant inserter recited in claim 13 wherein said implant body further includes an anterior interior compartment, a posterior interior compartment, an internal partition separating said anterior interior compartment from said posterior interior compartment, a passage through said partition coaxial with said central longitudinal axis establishing communication between said anterior interior compartment and said posterior interior compartment, an anterior bore extending through said anterior end wall coaxial with said central longitudinal axis and in communication with said anterior interior compartment, a posterior bore extending through said posterior end wall coaxial with said central longitudinal axis and in communication with said posterior interior compartment, an anterior window in each of said superior and inferior sides respectively in communication with said anterior interior compartment, and a posterior window in each of said superior and inferior sides respectively in communication with said posterior interior compartment, said anterior windows being located between said anterior end wall and said internal partition, said posterior windows being located between said posterior end wall and said internal partition, said anterior windows and said posterior windows creating discontinuity in at least some of said ridges and said grooves along each of said superior and inferior sides.

15. An intervertebral implant and implant inserter, comprising
an intervertebral implant comprising an implant body including superior and inferior sides, opposed medial and lateral sides, an anterior end wall, and a posterior end wall, each of said superior and inferior sides having a straight central section and convexly curving side sections on opposite sides of said central section, each of said side sections being composed of a plurality of convex surface segments of different radii of curvature including a first convex surface segment extending from said central section, a second convex surface segment extending from said first convex surface segment, and a third convex surface segment extending from said second convex surface segment to meet the corresponding one of said medial or lateral sides, each of said medial and lateral sides having a concave inward portion and superior and inferior concave outward portions extending from said concave inward portion to meet said third convex surface segments of the corresponding ones of said superior and inferior sides, each of said medial and lateral sides having a cavity defined by the corresponding ones of said concave inward portion and said superior and inferior concave outward portions; and
an implant inserter comprising a pair of holding members configured to be respectively removably received in said cavities with a mating fit, each of said holding members having a cross-sectional configuration defined by a convex inner portion complementary to the corresponding one of said concave inward portions of said implant, and a convex outer portion including an intermediate convex section extending between said third convex surface segments of said implant when said holding member is matingly received in a corresponding one of said cavities, each of said intermediate convex sections being composed of a plurality of convex surface segments of different radii of curvature including upper and lower convex surface segments, intervening convex surface segments extending respectively from said upper and lower convex surface segments, and a middle convex surface segment extending between said intervening convex surface segments.

16. The intervertebral implant and implant inserter recited in claim 15 wherein said first convex surface segments have a first radius of curvature, said second convex surface segments have a second radius of curvature, and said third convex surface segments have a third radius of curvature, said first radius of curvature being greater than said second radius of curvature, and said third radius of curvature being greater than said first radius of curvature.

17. The intervertebral implant and implant inserter recited in claim 16 wherein said upper and lower convex surface segments have a radius of curvature less than said second radius of curvature, said intervening convex surface segments have a radius of curvature less than said radius of curvature of said upper and lower convex surface segments, and said middle convex surface segments have a radius of curvature less than said radius of curvature of said intervening convex surface segments.

18. The intervertebral implant and implant inserter recited in claim 15 wherein said second convex surface segments meet said third convex surface segments at straight edges extending between said anterior end wall and said posterior end wall, said third convex surface segments meet said outward portions of said medial and lateral sides at tips extending between said anterior end wall and said posterior end wall, and said third convex surface segments have a depth between said edges and said tips that taper from anterior to posterior direction.

19. The intervertebral implant and implant inserter of claim 18 wherein said implant body further includes a central longitudinal axis, an anterior interior compartment, a posterior interior compartment, an anterior opening in said concave inward portion of each of said medial and lateral sides in communication with said anterior interior compartment, and a posterior opening in said concave inward portion of each of said medial and lateral sides in communication with said posterior interior compartment.

20. The intervertebral implant and implant inserter recited in claim 18 wherein each of said tips has an anterior recess and a posterior recess in respective correspondence with said anterior opening and said posterior opening in each of said medial and lateral sides.
